(19) United States
(12) Patent Application Publication
(10) Pub. No.: US 2025/0345185 A1
(43) Pub. Date: Nov. 13, 2025

(54) ORTHOPEDIC INSTRUMENT CONNECTION MECHANISMS AND RELATED ASSEMBLIES AND SYSTEMS FOR PROVIDING PROVISIONAL FIXATION

(71) Applicant: MicroPort Orthopedics Holdings Inc., Arlington, TN (US)

(72) Inventors: Michael D. Hollandsworth, Broomfield, CO (US); Fred W. Bowman, Germantown, TN (US); Michael L. Brooks, Brighton, TN (US)

(73) Assignee: MicroPort Orthopedics Holdings Inc., Arlington, TN (US)

(21) Appl. No.: 19/201,014

(22) Filed: May 7, 2025

Related U.S. Application Data
(60) Provisional application No. 63/644,328, filed on May 8, 2024.

(51) Int. Cl. A61F 2/46; A61F 2/30; A61F 2/38

(52) U.S. Cl. CPC A61F 2/461; A61F 2/30724; A61F 2/30771; A61F 2/389; A61F 2002/30205; A61F 2002/30599; A61F 2002/30604; A61F 2002/3069; A61F 2002/3092

(57) ABSTRACT

An orthopedic medical device connection mechanism comprising: a first medical device comprising: a first body having: a nesting outer surface, a spacer recess, the spacer recess extending into the first body and communicating with the nesting outer surface, and a movable spacer disposed within the spacer recess, the movable spacer having an internal end internally disposed from an external end, wherein the external end is adjacently disposed to the nesting outer surface; and a second medical device comprising: a second body configured to be nested with the first body, a porous inner surface internally disposed from a second body outer surface, wherein the porous inner surface defines a connector area, and wherein the nesting outer surface is configured to be disposed within the concave connector area adjacent to the porous inner surface in an assembled configuration.

### RELATED APPLICATION

This application claims the benefit of co-pending U.S. Provisional Patent Application No. 63/644,328, filed on May 8, 2024. The disclosure of this related priority application is hereby incorporated into the present disclosure in its entirety.

### BACKGROUND OF THE INVENTION

### Technical Field

The present disclosure relates generally to the field of orthopedic surgery, and more particularly to an orthopedic instrument connection mechanism and related systems and assemblies.

### Related Art

In many minimally invasive orthopedic surgical procedures, the surgeon uses a variety of tools to prepare and orient endoprosthetic implants. Such instruments include a variety of broaching, reaming, and placement tools. To work effectively and to minimize the time that a patient is under anesthesia, the surgeon typically works with a team of technicians, nurses, and other medical professionals to assemble and prepare the various sterilized surgical instruments prior to use.

Some prior orthopedic medical device connection mechanisms consisted of single points of engagement. While these were generally quick to connect and disconnect, these designs could lead to inaccurate or lose fittings of the instruments. Other designs included delicate and complex spring and pin mechanisms, which could be prone to failure after repeated impaction.

### SUMMARY OF THE INVENTION

The problems of the prior art are solved by an orthopedic medical device connection mechanism comprising: a first orthopedic medical device comprising: a first body, the first body having a nesting outer surface, the nesting outer surface defining a first geometric shape, and an area defining a spacer recess, the spacer recess extending into the first body and communicating with the nesting outer surface, and a movable non-rigid spacer disposed within the spacer recess, the movable non-rigid spacer having an internal end internally disposed from an external end, wherein the external end is adjacently disposed to the nesting outer surface; and a second orthopedic medical device comprising: a second body configured to be nested with the first body, a porous inner surface internally disposed from a second body outer surface, the porous inner surface defining a second geometric shape, the second geometric shape being complimentary to the first geometric shape, wherein the porous inner surface defines a plurality of pores, wherein the nesting outer surface abuts the porous inner surface in an assembled configuration, and wherein the external end of the moveable non-rigid spacer is received by a pore of the plurality of pores in the porous surface in the assembled configuration.

It is contemplated that certain exemplary embodiments described herein may permit quick, accurate, and secure assembly and disassembly of orthopedic instruments, while permitting the connection mechanism components to survive repeated blunt force from impaction instruments.

It is further contemplated that certain exemplary embodiments described herein may permit the use of a variety of modular orthopedic medical device assemblies that can be configured to be assembled and disassembled via an exemplary medical device connection mechanism in accordance with this disclosure.

It is still further contemplated that certain exemplary embodiments described herein may permit the use of fewer manual orthopedic instruments at the time of surgery compared to prior designs that utilized complex connection mechanisms.

### BRIEF DESCRIPTION OF THE DRAWINGS

The foregoing will be apparent from the following more particular description of exemplary embodiments of the disclosure, as illustrated in the accompanying drawings. The drawings are not necessarily to scale, with emphasis instead being placed upon illustrating the disclosed embodiments.

FIG. 1 is a side perspective view of an exemplary first orthopedic medical device, wherein the first orthopedic medical device is an exemplary cone inserter.

FIG. 2 is a cross-sectional side vide of the exemplary first orthopedic medical device of FIG. 1 bisected along the plane A-A.

FIG. 3 is an expanded perspective view of an exemplary orthopedic insertion instrument assembly comprising an exemplary orthopedic medical device connection mechanism, depicted in a disassembled configuration.

FIG. 4 is an assembled perspective view of the exemplary orthopedic insertion instrument assembly of FIG. 3.

FIG. 5 is a cross-sectional side vide of the exemplary assembled orthopedic insertion instrument assembly of FIG. 4 bisected along the plane B-B.

FIG. 6 is a perspective view of an exemplary assembled orthopedic insertion instrument assembly being used to insert a second orthopedic medical device into a resected and broached proximal tibia.

FIG. 7 is a detailed close up view of the porous inner surface of an exemplary second orthopedic medical device.

FIG. 8 is a detailed close up cross-sectional side view of a non-rigid spacer of an exemplary first orthopedic medical device engaging a pore of the porous inner surface of an exemplary second orthopedic medical device in an assembled configuration.

### DETAILED DESCRIPTION OF THE PREFERRED EMBODIMENTS

The following detailed description of the preferred embodiments is presented only for illustrative and descriptive purposes and is not intended to be exhaustive or to limit the scope and spirit of the invention. The embodiments were selected and described to best explain the principles of the invention and its practical application. One of ordinary skill in the art will recognize that many variations can be made to the invention disclosed in this specification without departing from the scope and spirit of the invention.

Similar reference characters indicate corresponding parts throughout the several views unless otherwise stated. Although the drawings represent embodiments of various features and components according to the present disclosure, the drawings are not necessarily to scale and certain features may be exaggerated to better illustrate embodiments of the present disclosure, and such exemplifications are not to be construed as limiting the scope of the present disclosure.

Except as otherwise expressly stated herein, the following rules of interpretation apply to this specification: (a) all words used herein shall be construed to be of such gender or number (singular or plural) as such circumstances require; (b) the singular terms “a,” “an,” and “the,” as used in the specification and the appended claims include plural references unless the context clearly dictates otherwise; (c) the antecedent term “about” applied to a recited range or value denotes an approximation with the deviation in the range or values known or expected in the art from the measurements; (d) the words, “herein,” “hereby,” “hereto,” “hereinbefore,” and “hereinafter,” and words of similar import, refer to this specification in its entirety and not to any particular paragraph, claim, or other subdivision, unless otherwise specified; (e) descriptive headings are for convenience only and shall not control or affect the meaning of construction of part of the specification; and (f) “or” and “any” are not exclusive and “include” and “including” are not limiting. Further, the terms, “comprising,” “having,” “including,” and “containing” are to be construed as open-ended terms (i.e., meaning “including but not limited to”).

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether explicitly described.

To the extent necessary to provide descriptive support, the subject matter and/or text of the appended claims are incorporated herein by reference in their entirety.

Recitation of ranges of values herein are merely intended to serve as a shorthand method of referring individually to each separate value falling within the range of any sub-ranges there between, unless otherwise clearly indicated herein. Each separate value within a recited range is incorporated into the specification or claims as if each separate value were individually recited herein. Where a specific range of values is provided, it is understood that each intervening value, to the tenth or less of the unit of the lower limit between the upper and lower limit of that range and any other stated or intervening value in that stated range of sub range thereof, is included herein unless the context clearly dictates otherwise. All subranges are also included. The upper and lower limits of these smaller ranges are also included therein, subject to any specifically and expressly excluded limit in the stated range.

The terms, “horizontal” and “vertical” are used to indicate direction relative to an absolute reference, i.e., ground level. However, these terms should not be construed to require structure to be absolutely parallel or absolutely perpendicular to each other. For example, a first vertical structure and a second vertical structure are not necessarily parallel to each other.

Throughout this disclosure and unless otherwise noted, various positional terms, such as “distal,” “proximal,” “medial,” “lateral,” “anterior,” and “posterior,” will be used in the customary manner when referring to the human anatomy. More specifically, “distal” refers to the area away from the point of attachment to the body, while “proximal” refers to the area near the point of attachment to the body. For example, the distal femur refers to the portion of the femur near the tibia, whereas the proximal femur refers to the portion of the femur near the hip. The terms, “medial” and “lateral” are also essentially opposites. “Medial” refers to something that is disposed closer to the middle of the body. “Lateral” means that something is disposed closer to the right side or the left side of the body than to the middle of the body. Regarding, “anterior” and “posterior,” “anterior” refers to something disposed closer to the front of the body, whereas “posterior” refers to something disposed closer to the rear of the body.”

“Varus” and “valgus” are broad terms and include without limitation, rotational movement in a medial and/or lateral direction relative to the knee joint.

The term, “mechanical axis” of the femur refers to an imaginary line drawn from the center of the femoral head to the center of the distal femur at the knee.

The term, “anatomic axis” refers to an imaginary line drawn lengthwise down the middle of femoral shaft or tibial shaft, depending upon use.

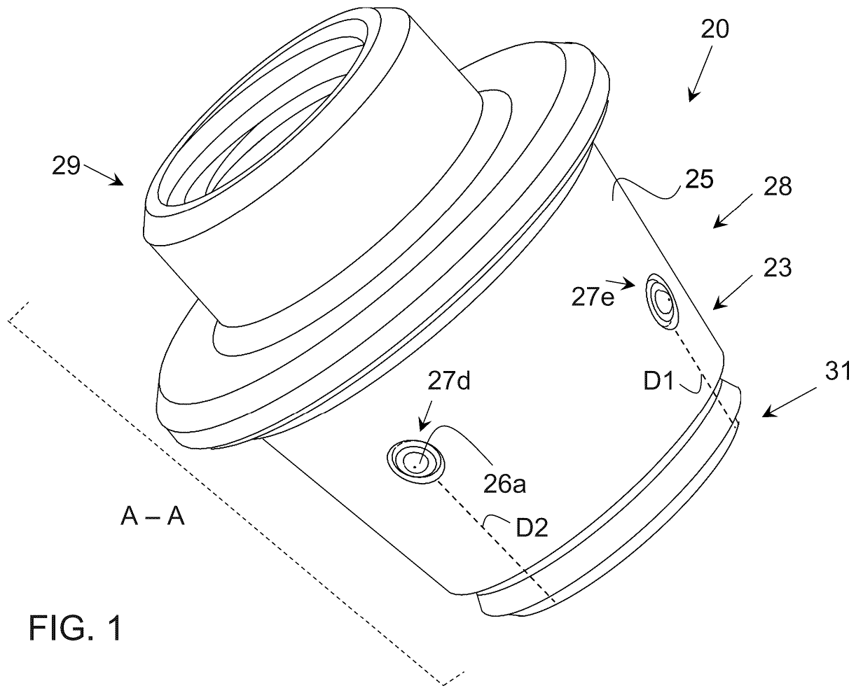

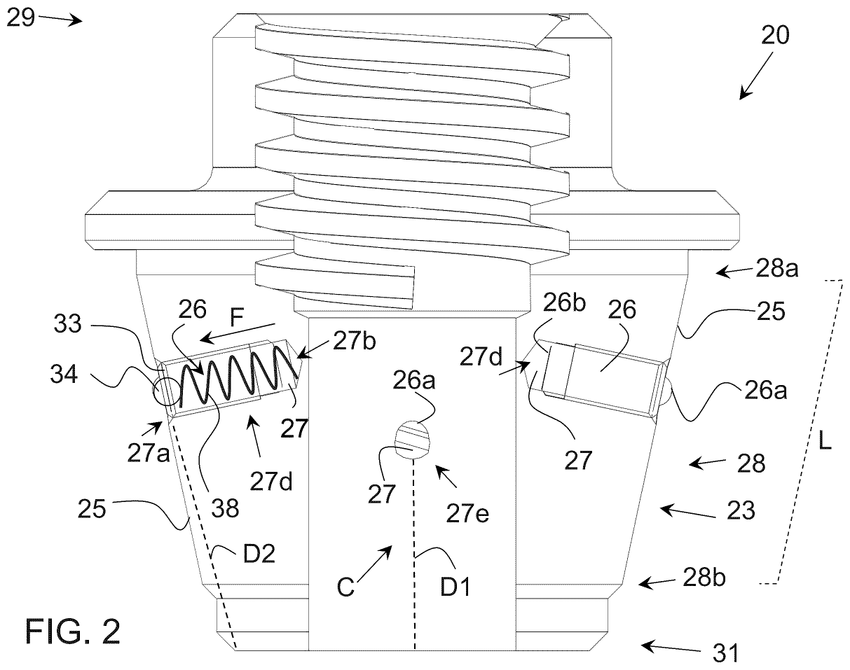

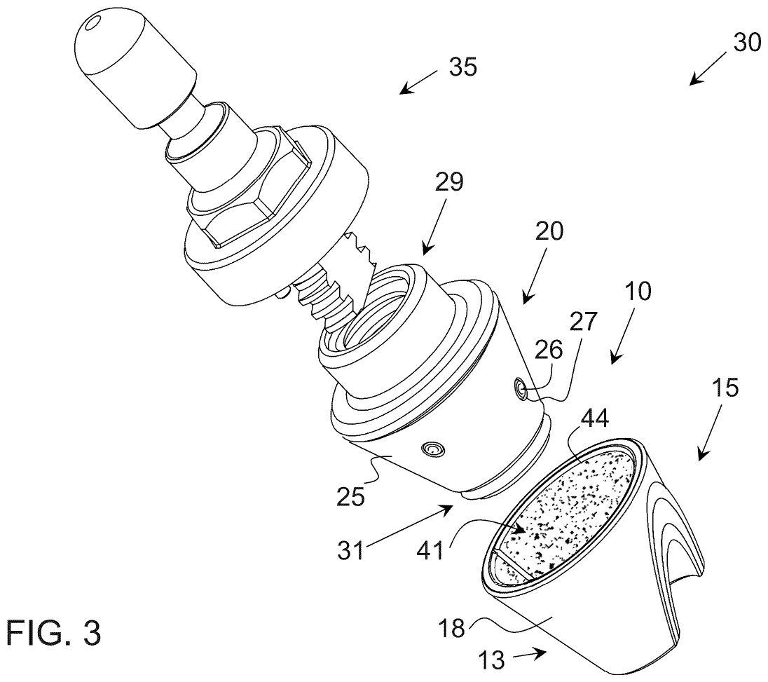

FIGS. 1-2 depict an exemplary first orthopedic medical device 20 of an exemplary orthopedic insertion instrument assembly 30 (FIGS. 3-6) comprising an exemplary medical device connection mechanism 10 (FIGS. 3-5). It will be appreciated that the orthopedic medical devices disclosed herein are generally contemplated to be used in surgical procedures, particularly in orthopedic surgical procedures. It is contemplated that in certain exemplary embodiments, the medical devices may be orthopedic instruments, orthopedic implants, orthopedic trial implants, orthopedic instrument adapters, combinations or components thereof; however, nothing in this disclosure limits the contemplated “orthopedic medical devices” to the examples provided. In the depicted exemplary embodiment, the first orthopedic medical device 20 of an exemplary orthopedic medical device connection mechanism 10 (FIG. 3).

As exemplified in FIGS. 1 and 2, the first orthopedic medical device 20 (e.g., a cone inserter) comprising a proximal end 29 that is proximally disposed from a distal end 31 and a first body 23 that extends between the proximal end 29 and the distal end 31. The first body 23 comprises nesting portion 28 having a nesting portion proximal side 28a that is proximally disposed from a nesting portion distal side 28b along a length L, and a nesting outer surface 25 extending along the length L. In the depicted embodiment, the nesting portion 28 generally comprises first geometric shape, which is a frustoconical shape that tapers from the nesting portion proximal side 28a to the nesting portion distal side 28b along the length L. However, all nesting shapes are considered to be within the scope of this disclosure.

By way of example, nested shapes can include nested conical shapes, nested polyhedral shapes, and nested dome shapes. Example nested conical shapes include: cones, frustums of cones, elliptic cones, frustums of elliptic cones, portions thereof, combinations thereof, and permutations thereof. Examples of nested polyhedral shapes include: pyramids, frustums of pyramids, wedges, prisms, cupolae, frustums of cupolae, portions thereof, combinations thereof, and permutations thereof. Examples of nested dome shames include: hemispheres, frustums of hemispheres, domes, frustums of domes, domes of spheroids, frustums of domes of spheroids, domes of ellipsoids, frustums of domes of ellipsoids, portions thereof, combinations thereof, and permutations thereof. It will be appreciated that combinations and permutations of any nested conical shape with any nested polyhedral or nested dome shape is considered to be within the scope of the disclosure.

Referring back to the nesting portion 28 of FIGS. 1 and 2, the nesting distal side 28b is disposed closer to the distal end 31 of the first orthopedic medical device 20 than the nesting proximal side 28a. The nesting portion 28 of the first body 23 and the nesting outer surface 25 defines a spacer recess 27 extending into the nesting portion 28 of the first body 23. That is, a first end (or external end) 27a of the spacer recess 27 communicates with and is adjacently disposed to the nesting outer surface 25 and a second end (or internal end) 27b of the spacer recess 27 is internally disposed from the first end 27a of the spacer recess 27 relative to a centerline C extending vertically through the first orthopedic medical device 20 such that the spacer recess 27 extends between the first end 27a and second end 27b and such that at least a portion of the spacer recess 27 is disposed in the nesting portion 28 of the first body 23 of the first orthopedic medical device 20.

In the depicted embodiment, the spacer recess 27 is disposed substantially perpendicular to the nesting outer surface 25 that is disposed adjacent to the first end 27a of the spacer recess 27. It is contemplated that a spacer recess 27 disposed in this manner may be more efficient to machine compared to spacer recesses 27 disposed at other angles relative to the nesting outer surface 25 that is disposed adjacent to the first end 27a of the spacer recess 27. However, all other physically possible angles are considered to be within the scope of this disclosure. It is contemplated that an angle that results in a longer spacer recess 27 may accommodate a longer spring element 38, other biasing member, or spacer element 34 compared to those in the depicted embodiment.

A movable non-rigid spacer 26 is disposed within the spacer recess 27. The movable non-rigid spacer 26 has an internal end 26b internally disposed from an external end 26a. The movable non-rigid spacer 26 extends between the internal end 26b and the external end 26a. The external end 26a is adjacently disposed to the nesting outer surface 25. Non-limiting examples of a moveable non-rigid spacers 26 include moveable non-rigid spacer assemblies, such as a ball plunger assembly, a bear bearing with a garter spring, or a retractable pin. Another examples of a moveable non-rigid spacer 26 includes an o-ring.

FIG. 2 details a representative example of a moveable non-rigid spacer 26, wherein the moveable non-rigid spacer 26 is a ball plunger assembly. In the depicted example, the moveable non-rigid spacer assembly 26 comprises a spring element 38 (e.g., a helical spring) and a spacer element 34 (e.g., a ball of the ball plunger, or ball bearing). The spring element 38 is disposed within the spacer recess 27. The spacer element 34 is disposed at the first end 27a of the spacer recess 27 such that the spacer element 34 is partially inside the spacer recess 27 and partially outside of the spacer recess 27. An annular retainer 33 is disposed at the first end 27a of the spacer recess 27. The annular retainer 33 encircles an opening having an opening diameter that this less than the diameter of the spacer element 34. In this manner, the annular retainer 33 prevents the spacer element 34 from exiting the spacer recess 27 from the first end 27a completely. The spring element 38 imparts a spring force F on the spacer element 34. The selected spring element 38 and orientation of the spacer recess 27 orients the spring force F towards the nesting outer surface 25.

It is contemplated that in certain exemplary embodiments, the moveable non-rigid spacers 26 can be designed to be reusable components or they can be designed to be disposable or for limited use. The non-rigid spacers 26 or components thereof can be made from clinically proven biocompatible materials of sufficient hardness to prevent erosion or wear of the material during normal use. Examples of such materials include stainless steel, and titanium. It is further contemplated that components of disposable or limited use non-rigid spacers 26 can be made from polyether ether ketone (“PEEK”), polyethylene (“PE”), including but not limited to ultra-high molecular weight polyethylene (“UHMWPE”), and cross-linked polyethylene (“XLPE”), and polyamide (including but not limited to a glass-filled polyamide and a carbon fiber filled polyamide).

Exemplary embodiments in accordance with the present disclosure may comprise a first orthopedic medical device 20 having multiple areas defining spacer recesses 27. A non-rigid spacer 26 is desirably placed in each of the multiple spacer recesses 27. In other exemplary embodiments, multiple non-rigid spacers 26 can be placed in a spacer recess 27. In certain exemplary embodiments, the multiple spacer recesses 27 can be evenly distributed around first body 23. In other exemplary embodiments, the multiple spacer recesses 27 can be unevenly distributed around the first body 23.

The exemplary orthopedic insertion instrument assembly (or components thereof) 30 depicted in FIGS. 1-6 comprises four spacer recesses 27, wherein each spacer recess 27 is disposed about 90 degrees (°) from each adjacent spacer recess 27. The four depicted spacer recesses 27 comprise two pairs of opposing spacer recesses 27d, 27e. A first pair of opposing spacer recesses 27e is disposed closer to the distal end 31 of the first orthopedic medical device 20 than the second pair of opposing spacer recesses 27d. Stated differently, the first pair of opposing spacer recesses 27e is disposed at a first distance D1 from the distal end 31 of the first orthopedic medical device 20 and a second pair of opposing spacer recesses 27d is disposed at a second distance D2 from the distal end 31 of the first orthopedic medical device 20, wherein the first distance D1 does not equal the second distance D2. The first distance D1 and the second distance D2 represent the shortest distance from the first ends 27a of the respective spacer recesses 27 to the distal end 31 of the first orthopedic medical device 20. In other exemplary embodiments, the spacers recesses 27 can be at the same height throughout. In yet other exemplary embodiments, spacer recesses 27 can be disposed at a third distance, a fourth distance, a fifth distance, or further distances relative to prior distances provided that the additional distances do not equal the prior distances in measurement value. Combinations and permutations of the forgoing are considered to be within the scope of this disclosure.

Without being bound by theory, it is contemplated that having spacer recesses 27 and corresponding non-rigid spacers 26 disposed around the first orthopedic medical device 20 at different heights (e.g., an embodiment in which at least one spacer recesses 27 and non-rigid spacer 26 is disposed closer to the distal end 31 of the first orthopedic medical device 20 than other spacer recesses 27 containing non-rigid spacers 26) can be desirable for use in orthopedic insertion instrument assemblies 30 that are likely to be bumped or jostled during normal use. Applicant has found that spacing multiple spacer recesses 27 and their corresponding non-rigid spacers 26 at different heights along the first body 23 surprisingly and unexpectedly strengthened the provisional fixation bond and that the second orthopedic medical device 15 far less likely to become dislodged or disengaged from the first orthopedic medical device 20 through inadvertent bumps or through vigorous handling when used together with a porous inner surface 44 as described further infra.

FIG. 3 is an expanded perspective view of an exemplary orthopedic insertion instrument assembly 30 having an exemplary orthopedic medical device connection mechanism 10 depicted in a disassembled configuration. The exemplary orthopedic medical device connection mechanism 10 comprises the first orthopedic medical device 20 (e.g., a cone inserter) configured to provisionally affix to a second orthopedic medical device 15 (e.g., a cone implant) and an additional orthopedic medical device 35 (e.g., a broach adapter) configured to selectively engage a proximal end 29 of the first orthopedic medical device 20.

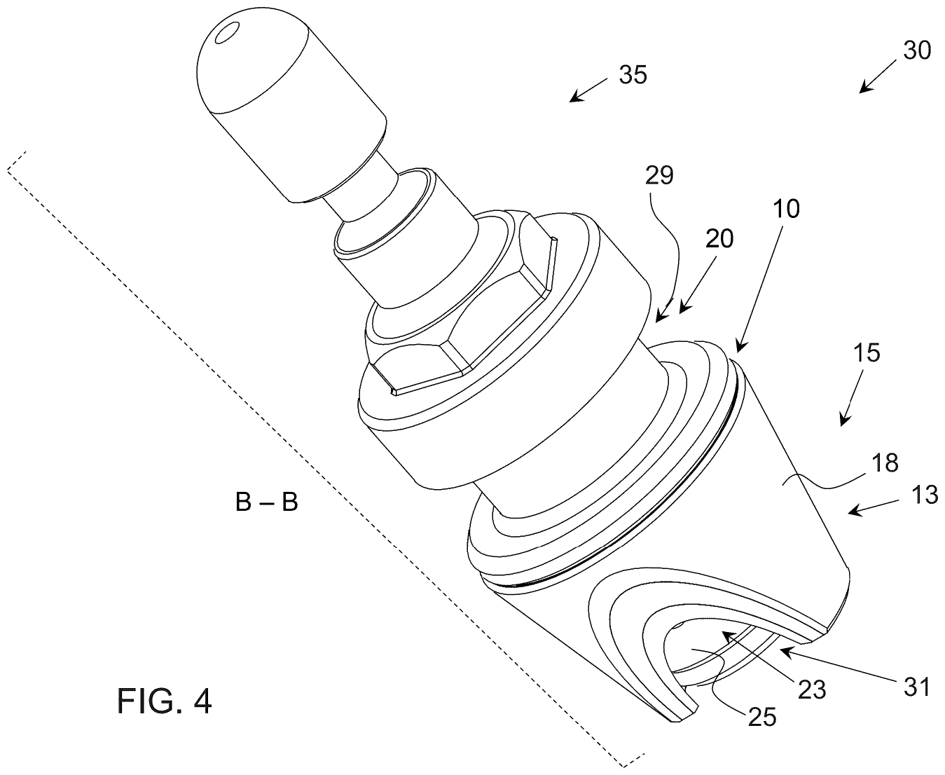

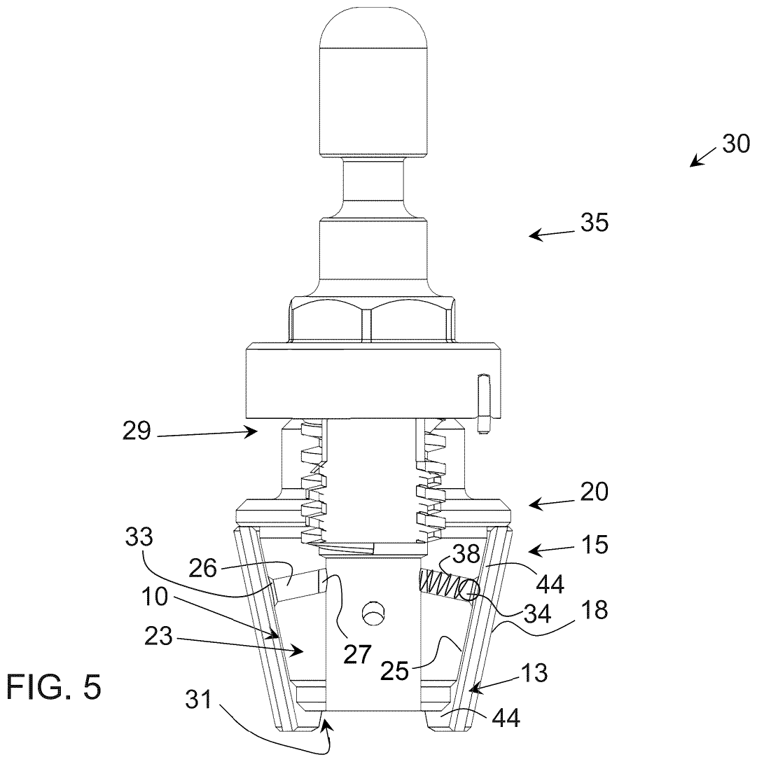

The second orthopedic medical device 15 comprises a second body 13 configured to be nested with the first body 23. The second body 13 comprises a porous inner surface 44 internally disposed from a second body outer surface 18. In the depicted embodiment, the porous inner surface 44 defines a concave area 41. The nesting outer surface 25 is configured to be closely received by the concave area 41 and disposed adjacent to the porous inner surface 44 in an assembled configuration (see FIGS. 4 and 5).

It will be appreciated that a second body 13 that is, “configured to be nested” comprises an inner surface (see 44) of the second body 13 that is closely dimensioned to abut a nesting outer surface 25 of the nesting portion 28 of the first body 23 along at least a portion of a length L (FIG. 2) of the outer surface (see 25) of the nesting portion 28 of the first body 23, or vice versa, such that the receiving inner surface (see 44) defines a concave area 41, and such that the inserting outer surface (see 25) abuts the receiving inner surface (see 44) substantially along the majority of a perimeter area of the receiving surface (see 44, see FIGS. 3 and 5).

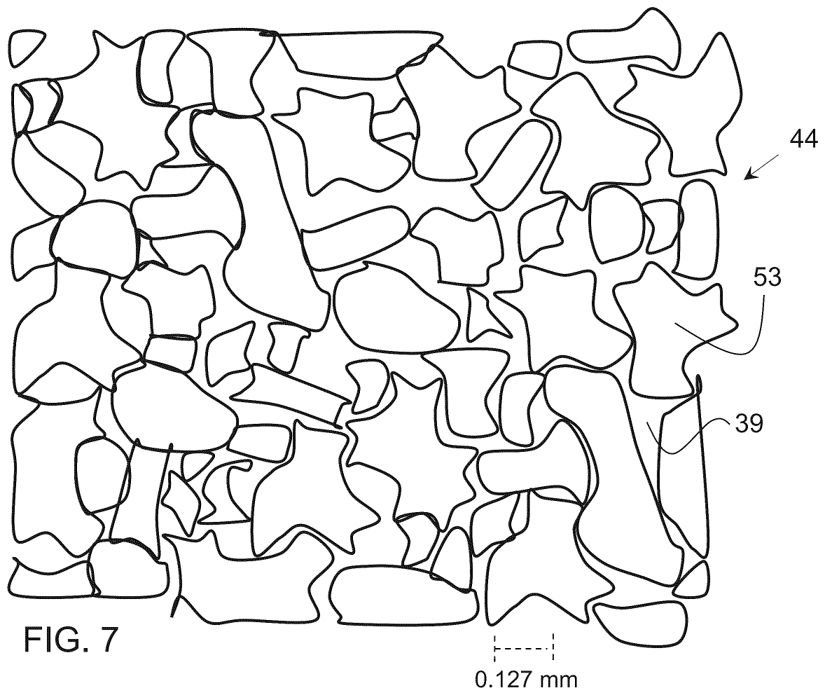

FIG. 7 is a close up view of an exemplary porous inner surface 44. All porous inner surfaces 44 having a mean pore diameter that is slightly greater than the exposed diameter of the non-rigid spacer 26 is considered to be within the scope of this disclosure, because this engagement feature, either taken by itself or in combination with the other engagement features disclosed herein, such as the closely adjacent disposal of the nesting outer surface 25 to the porous inner surface 44 in the assembled configuration, are believed to facilitate resilient provisional fixation in orthopedic instruments. A porous surface 44 may comprise a complex three-dimensional microstructure (as is often created through diffusion bonding) or a simple three-dimensional microstructure (as in the laser sintering of beads of fairly uniform diameter). However, porous surfaces 44 are typically characterized by having a three-dimensional scaffolding 53 defining a plurality of pores 39 surrounding the scaffolding 53.

One such suitable porous surface 44 is described in “Mechanical Characteristics of OsteoSync™ Ti” published by Sites Medical Research and Development, and which is incorporated herein by reference. In exemplary embodiments, the mean porosity of the porous inner surface 44 (i.e., the fraction of the volume of the pores 39 over the volume of the entire porous surface 44) desirably exceeds 50%. The inner porous surface 44 can comprise titanium, a titanium alloy, cobalt chrome, cobalt chrome alloys, or other clinically proven biocompatible material having low mass loss due to abrasion. The mean diameter of the pores 39 in the porous inner surface 44 desirably range from about 400 micrometers (“μm”) to about 800 μm. In some exemplary embodiments, the mean pore diameter can be about 523 μm with a standard deviation of 0.0021 (53 μm), as determined by calculating the mean void intercept length. The mean void intercept length is determined by superimposing measurement grid lines parallel to a porous inner surface 44 substrate in a field. The average length of the line segments overlaying the void space is the mean void intercept length for that field, which is a representative measure of the scale, or size, of the pores in a porous structure. In other exemplary embodiments, the porous surface 44 can be multi-layered. In such exemplary embodiments, the outermost or proximal-most layer may be configured to interface with bone or other tissue when surgically implanted into a patient. The patient's bone or other tissue can grow through the pores 39 over time to improve fixation of the implant in the patient's bone or other tissue. In such multi-layered embodiments, the mean pore diameter of the tissue interface layer can be about 1280 μm, with a standard deviation of 0.229 (582 μm), a maximum of about 0.1262 (3205 μm) and a minimum of about 0.0243 (617 μm).

The coefficient of friction for the porous surface 44 should desirably be between 0.75 and 1.20 and should even more desirably exceed 1.0 when the following criteria are applied: the porous surface 44 should first be coupled to a 10 pound per cubic foot (“PCF”) or 160.184 kilograms per square meter (“kg/m³”) sawbone simulated bone and then a horizontal displacement should be applied to the simulated bone at a constant rate. The resulting frictional force should be recorded. The friction coefficient will be the peak friction force divided by the nominal normal force. The mechanical stiffness of the porous surface 44 (i.e., in both tension and shear) desirably exceeds 6 gigapascals (“GPa”).

There are several different processes for manufacturing porous surfaces for use in orthopedic medical devices. These include diffusion bonding, chemical vapor deposition, sintering, and additive manufacturing. These porous surfaces 44 can be manufactured independently of the second orthopedic medical device 15 and then be metallurgically attached to the second orthopedic medical device 15, or the porous surface 44 can be created as the second orthopedic medical device 15 is being created (e.g. especially in an additive manufacturing process).

The inventors have unexpectedly discovered that a porous inner surface 44, including porous inner surfaces 44 with the above-described properties, when used with non-rigid spacers 26 disposed in the body 23 of a nesting portion 28 of a first orthopedic medical device 20 permits resilient provisional fixation of the first orthopedic medical device 20 to the second orthopedic medical device 15 in the assembled configuration.

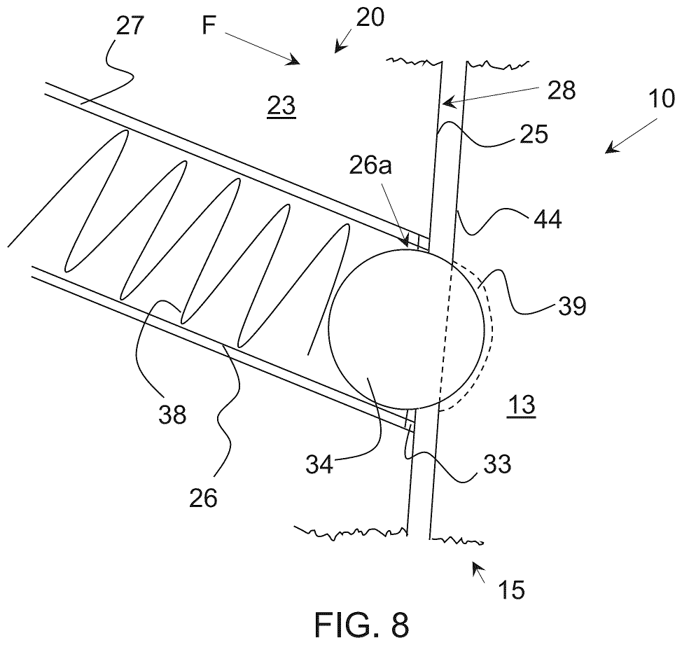

Without being bound by theory, it is contemplated that a porous inner surface 44 having a mean pore diameter that is slightly greater than the exposed diameter of the non-rigid spacer 26 is desirable because the likelihood that a given pore 39 of the porous inner surface 44 will closely receive the spacer element 34 (e.g., a ball of a ball plunger) of the non-rigid spacer 26 is greatly increased over embodiments having a mean pore diameter substantially greater or smaller than the exposed diameter of the non-rigid spacer 26 (see FIG. 8). In this manner, it is contemplated that the coefficient of friction between the engagement surfaces of the first orthopedic medical device 20 and the second orthopedic medical device 15 is further increased and resilient provisional fixation can be more effectively achieved.

In an exemplary embodiment, the porous inner surface 44 can be primarily located in areas of the concave area 41 that are more likely to receive the external end 26a of the non-rigid spacer 26 when provisionally engaged to the nesting portion 28 of the first body 23 in the assembled configuration.

In certain exemplary embodiments, the porous inner surface 44 can be substantially smooth (i.e., the scaffolding 53 can comprise a smooth surface). In other exemplary embodiments, the porous inner surface 44 can be substantially roughened (i.e., the scaffolding 53 can comprise a roughened surface).

In certain exemplary embodiments, the second body outer surface 18 can be porous, roughened, or substantially smooth depending upon the desired use for the second medical device 15. In applications in which the second medical device 15 is designed to be left in a patient's body for prolonged periods, the second body outer surface 18 may be porous to accommodate bone ingrowth for more permanent fixation. A porous outer surface 18 may be the same type of porous surface as the porous inner surface 44. In other exemplary embodiments, the porous outer surface 18 may have different physical properties than the porous inner surface 44.

FIGS. 4-5 depict the exemplary orthopedic insertion instrument assembly 30 of FIG. 3 in the assembled configuration. FIG. 5 is a cross-sectional side view that emphasizes the provisional fixation of the first orthopedic medical device 20 to the second orthopedic medical device 15. “Provisional fixation” describes the loose engagement between the first orthopedic medical device 20 and the second orthopedic medical device 15 in the assembled configuration.

In the depicted cross-sectional side view, the second body 13 is nested with the first body 23. The porous inner surface 44 is internally disposed from a second body outer surface 18 (see also FIG. 8). The porous inner surface 44 defines a second geometric shape, which is complimentary to the first geometric shape defined by the nesting portion 28 of the nesting outer surface 25. In the depicted assembled configuration, the nesting outer surface 25 of the nesting portion 28 abuts the porous inner surface 44 along substantially the entire length L of the nesting portion 28. In other exemplary embodiments, the nesting outer surface 25 of the nesting portion 28 may abut the porous inner surface 44 along less than the entire length of the nesting portion 28. As seen more clearly in FIG. 8, an external end 26a (see also 34) of the moveable non-rigid spacer 26 is received by a pore 39 of the porous inner surface 44.

FIG. 8 is a detailed close up cross-sectional side view of an exemplary connection mechanism 10 comprising a non-rigid spacer 26 of an exemplary first orthopedic medical device 20, a pore 39 of the porous inner surface 44 of an exemplary second orthopedic medical device 15, and the nesting outer surface 25 of the nesting portion 28 abutting the porous inner surface 44. The exemplary connection mechanism 10 is depicted in an assembled configuration.

The force F of the spring 38 biases the ball plunger 34 outwardly from the non-rigid spacer 26 into a pore 39 of the porous inner surface 44 of the second medical device 15. Although a gap is shown between the outer surface 25 of the nesting portion 28, and the porous inner surface 44, it will be appreciated that this gap is exaggerated in the figure to highlight the non-rigid spacer 26 and one mechanism of engagement. It is contemplated that in practice, the outer surface 25 of the nesting portion 28 physically abuts the porous inner surface 44. The fiction between the outer surface 25 of the nesting portion 28 and the porous inner surface 44 is thought to contribute to the provisional fixation of the respective orthopedic medical devices 20, 15.

Without being bound by theory, it is contemplated that the frictional force between the nesting outer surface 25 and the porous inner surface 44 and the spring force F exerted by the external ends 26a of the non-rigid spacers 26 on the porous inner surface 44 (and preferably force F exerted through multiple pores 39 of the porous inner surface 44) are sufficient to engage the second orthopedic medical device 15 to the first orthopedic medical device for the purposes of light handling of an orthopedic insertion instrument assembly 30 comprising the exemplary connection mechanism 10.

Although the depicted embodiments show the spacer recesses 27 and non-rigid spacers 26 as being disposed in the first body 23, it will be appreciated that in other exemplary embodiments, the spacer recesses 27 and non-rigid spacers 26 can be disposed in the second body 13 such that the second end 27b of the spacer recess 27 communicates with the porous inner surface 44 and the internal end 26b of the non-rigid spacer 26 is proximally disposed to the porous inner surface 44. It will be further appreciated that combinations of the present embodiment and the embodiment described with reference to FIGS. 1-8 are considered to be within the scope of this disclosure.

Without being bound by theory, it is contemplated that arrangements in accordance with this disclosure are desirable for orthopedic insertion instrument assemblies 30 because the nested elements permit quick and sufficiently secure assembly with minimal need for visualization. If the circumference of the nesting outer surface 25 and the complementary porous inner surface 44 are circular, the first orthopedic medical device 20 can be inserted into the second orthopedic medical device 15 agnostic of radial orientation. If the circumference of the nesting outer surface 25 and the complementary porous inner surface 44 are ellipsoid, or if the respective perimeters comprise some other shape, the nested surfaces 25, 44 compel correct alignment as a condition for engagement. It is contemplated that this correct alignment can be achieved by feel and by the respective nested surfaces 25, 44 naturally finding their lowest energy state of abutment.

Once the second orthopedic medical device 15 has been inserted into the desired surgical area (e.g., into a broached area of a target bone), the friction of the surrounding area should surpass the provisional engagement friction holding the first orthopedic medical device 20 to the second orthopedic medical device 15. The surgeon can then tap an exposed side of the orthopedic insertion instrument assembly 30 to remove the first orthopedic medical device 20 from the second orthopedic medical device 15.

It is further contemplated that the exemplary connection mechanisms 10 disclosed herein can survive repeated dislodging impaction forces, while permitting quick assembly and disassembly of orthopedic medical devices having the mating components of the connection mechanism 10, which can ultimately contribute to a reduction in patient time under anesthesia.

Referring collectively to the particular embodiments of FIGS. 1-8, these particular examples depict components of an orthopedic insertion instrument assembly 30 configured for use in a knee arthroplasty procedure. However, it will be appreciated that other exemplary orthopedic instrument assemblies that are within the scope of this disclosure may be adapted for other orthopedic uses. A non-limiting list of other example applications of the exemplary orthopedic instrument assemblies include inserter assemblies adapted for use in other bones (e.g., femoral cone inserter assemblies, provisional orthopedic screw holders (such as intramedullary screws, pedicle screws, etc.) acetabular cup inserter assemblies, glenoid inserter assemblies, augment insert assemblies (for endoprosthetic implants, e.g., acetabular augments, tibial augments, femoral augments, etc.), and humeral cone inserter assemblies).

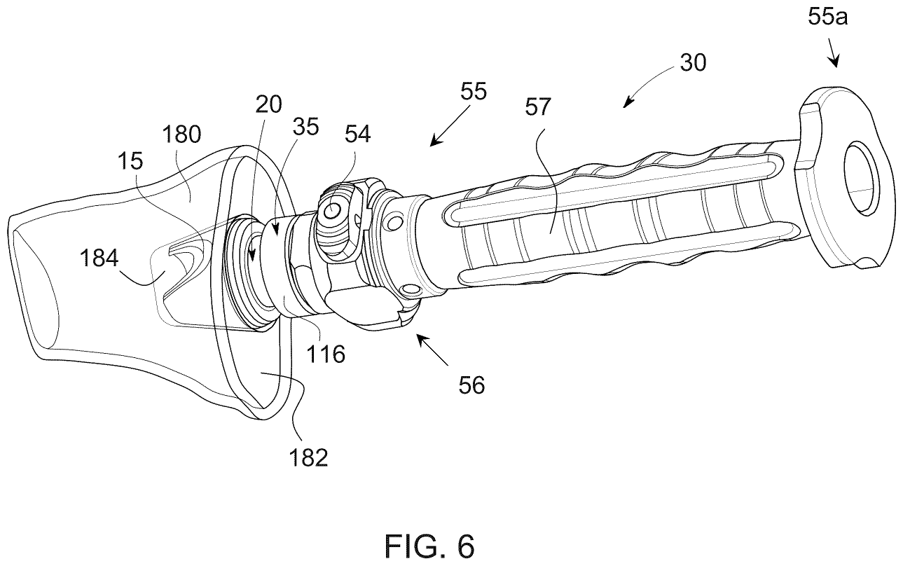

Briefly, in a typical knee arthroplasty procedure, the surgeon makes a generally vertical medial parapatellar incision of about five to six inches (or 12.7 centimeters (“cm”) to 15.24 cm) in length on the anterior or anteromedial aspect of the knee. The surgeon then continues to incise the fatty tissue to expose the anterior or anteromedial aspect of the joint capsule. The surgeon may then perform a medial parapatellar arthrotomy to pierce the joint capsule. A retractor may then be used to move the patella generally laterally (roughly about 90 degrees) to expose the distal condyles of the femur and the cartilaginous meniscus resting on the proximal tibial plateau. The surgeon then removes the meniscus and uses instrumentation to measure and resect the distal femur and proximal tibia 180 (FIG. 6) to accommodate trial—and eventually final—implants.

In some cases, the proximal tibia 180 or the distal femur presents voids and sections of poor bone quality that would compromise the overall stability of the final endoprosthetic implant construct. In these cases, surgeons can use conical implants (e.g., an example second orthopedic medical device 15) to fill and reinforce these sections of the bone to help prevent further bone degradation and improve the structural integrity of the bone surrounding the implant components.

Preparation of the proximal tibia 180 will be used as an example for the purposes of describing the general preparation and insertion of a bone reenforcing implant. To prepare the resected tibia 180 for the tibial components of an endoprosthetic knee implant, the surgeon generally resects the proximal tibia 180 in a planar manner to expose epiphyseal or metaphyseal marrow 182. The surgeon then inserts a series of progressively larger broaches into the proximal tibial metaphysis and diaphysis to create a cavity 184 in the bone marrow that mirrors the shape and size of the selected conical implant or implants. Broaching instrumentation, such as the instrumentation disclosed in U.S. patent application Ser. No. 18/612,382, and which is incorporated herein by reference, can include one or more modular broaches with mating attachment geometries and the adapter 35 that attaches to a handle assembly 55.

Once the cavity 184 has been prepared, the tibial cone implant (e.g., an example second orthopedic medical device 15) can be inserted into the tibial cavity 184 using an exemplary cone inserter instrument assembly 30 depicted in FIG. 6. The depicted exemplary cone inserter instrument assembly 30 comprises the first orthopedic medical device 20 (e.g., a cone inserter), the second orthopedic medical device 15 (e.g., a cone implant), the exemplary orthopedic medical device connection mechanism 10, and the additional orthopedic medical device 35 (e.g., a broach adapter) configured to selectively engage a proximal end 29 of the first orthopedic medical device 20 as described above with reference to FIGS. 3-5, and 8. The exemplary cone inserter instrument assembly 30 depicted in FIG. 6 further comprises a handle assembly 55 selectively engaged to the broach adapter 35. The handle assembly 55 comprises a handle 57 engaged to a release assembly 56. The release assembly 56 comprises a release button 54. A user can press the release button 54 to disengage the handle assembly 55 from the broach adapter 35.

An exemplary inserter instrument assembly 30 such as the one depicted in FIG. 6 permits a user to orient and insert the second orthopedic medical device 15 at a desired location in the proximal tibia 180 quickly. As the second orthopedic medical device 15 achieves biological fixation within the bone, the friction between the second body outer surface 18 and the metaphyseal bone exceeds the frictional force between the nesting outer surface 25 of the nesting portion 28 and the porous inner surface 44 and the spring force F between the moveable non-rigid spacers 26 and the porous inner surface 44. In this manner, the first orthopedic medical device 20 can be easily removed from the implanted second orthopedic medical device 15 without modifying the position of the implanted second medical device 15. To facilitate quick removal, the surgeon or technician may strike the proximal end 55a of the handle assembly 55 with a hammer or other blunt instrument to dislodge the connection mechanism 10.

A non-limiting list of example orthopedic implants include, but is not necessarily limited to: tibial cones, femoral cones, acetabular cups, glenoid cups, tibial, femoral, talar, scaphoid, lunate, metatarsal, metacarpal, phalangeal, pelvic, spinal, mandibular, humeral, radial, ulnar, or scapular components of endoprosthetic implants, and trial implants of any of the foregoing.

Other common orthopedic implants include implants or components thereof that extend into the metaphyseal or diaphyseal bone when implanted, other arguments, spacing elements, and void fillers. It will be appreciated that nothing in this disclosure limits the scope of this disclosure to the knee joint. All orthopedic instruments, orthopedic implants, and secondary adapters having an exemplary connection mechanism 10 are considered to be within the scope of this disclosure.

Components of an exemplary orthopedic insertion instrument assembly 30 can be provided in the form of a surgical kit. The components of the kit are preferably arranged in a convenient format, such as in a surgical tray or case. However, the kit components do not have to be packaged or delivered together, provided that they are assembled or collected together in the operating room for use at the time of surgery.

An exemplary kit can include any suitable embodiment of an exemplary orthopedic insertion instrument assembly 30, variations of the exemplary orthopedic insertion instrument assemblies 30 described herein, and any other exemplary orthopedic instrument assembly according to an embodiment. While it is contemplated that an exemplary kit may include one or more first orthopedic medical devices 20 (preferably of different sizes), one or more second orthopedic medical devices 15 (preferably of different sizes) and one or more additional orthopedic medical devices 35 (preferably of different sizes), it will be appreciated that certain kits may lack some or all of these elements.

Any suitable embodiment of a first orthopedic medical device 20, variations of the first orthopedic medical devices 20 described herein, and any other first orthopedic medical device 20 according to an embodiment, are considered to be within the scope of this disclosure. Any suitable embodiment of a second orthopedic medical device 15, variations of second orthopedic medical devices 15 described herein, and any other second orthopedic medical device 15 according to an embodiment are considered to be within the scope of this disclosure. Any suitable embodiment of an additional orthopedic medical device 35, variations of the additional orthopedic medical devices 35 described herein, and any other additional orthopedic medical devices 35 according to an embodiment, are considered to be within the scope of this disclosure.

Selection of a suitable number or type of first orthopedic medical device 20, second orthopedic medical device 15, and additional orthopedic medical devices 35 to include in a kit according to a particular embodiment can be based on various considerations, such as the procedure intended to be performed using the components included in the kit.

An exemplary orthopedic medical device connection mechanism comprises: a first orthopedic medical device comprising: a proximal end proximally disposed from a distal end; a first body disposed between the proximal end and the distal end, the first body having: a nesting portion disposed between the proximal end and the distal end of the first body, the nesting portion having a nesting portion proximal side proximally disposed from a nesting portion distal side along a length, and a nesting portion outer surface extending along the length, the nesting outer surface defining a first nesting geometric shape, an area defining a spacer recess, the spacer recess extending into the first body and communicating with the nesting portion outer surface, and a movable non-rigid spacer disposed within the spacer recess, the movable non-rigid spacer having an internal end internally disposed from an external end, wherein the external end is adjacently disposed to the nesting portion outer surface; and a second orthopedic medical device comprising: a second body configured to be nested with the first body, a porous inner surface internally disposed from a second body outer surface, the porous inner surface defining a second nesting geometric shape, the second nesting geometric shape being complimentary to the first nesting geometric shape, wherein the porous inner surface defines an plurality of pores, wherein the nesting portion outer surface of the first orthopedic medical device abuts the porous inner surface of the second orthopedic medical device in an assembled configuration, and wherein the external end of the moveable non-rigid spacer is received by a pore of the plurality of pores in the assembled configuration.

An exemplary orthopedic medical device connection mechanism assembly comprises: a first orthopedic medical device comprising: a first conical body, the first conical body having a nesting outer surface, and an area defining a spacer recess, the spacer recess extending into the first conical body and communicating with the nesting outer surface, and a movable non-rigid spacer disposed within the spacer recess, the movable non-rigid spacer having an internal end internally disposed from an external end, wherein the external end is adjacently disposed to the nesting outer surface; and a second orthopedic medical device comprising: a second conical body configured to closely receive the first conical body, a porous inner surface internally disposed from a second conical body outer surface, wherein the porous inner surface defines a concave area.

An exemplary orthopedic device connection mechanism assembly in an assembled configuration can be further characterized by the first conical body being disposed within the concave area, the first conical body being closely received by the second conical body in the concave area, and the external end of the movable non-rigid spacer engaging a pore defined by the porous inner surface in the assembled configuration.

In an exemplary orthopedic device connection mechanism assembly, the first medical device is an inserter selected from the group consisting essentially of: a femoral cone inserter, a provisional orthopedic screw holder, an acetabular cup inserter, a glenoid inserter, an augment inserter, and a humeral cone inserter assembly.

In an exemplary orthopedic device connection mechanism assembly, the second medical device is an implant selected from the group consisting essentially of: a tibial cone, a femoral cone, an acetabular cup, a glenoid cup, a tibial base implant, a femoral condylar implant, a talar implant, a scaphoid implant, a lunate implant, a metatarsal implant, a metacarpal implant, a phalangeal implant, a pelvic implant, a spinal implant, a mandibular implant, a humeral implant, a radial implant, an ulnar implant, or a scapular implant, components of any of the foregoing, trial implants of any of the foregoing, and components of trial implants of any of the foregoing.

In an exemplary orthopedic device connection mechanism assembly, the first conical body further defines multiple spacer recesses extending into the first conical body, wherein each of the multiple spacer recesses communicates with the nesting outer surface. In certain exemplary embodiments, the exemplary orthopedic device connection mechanism assembly further comprises multiple movable non-rigid spacers, wherein each movable non-rigid spacer of the multiple movable non-rigid spacers is disposed within a spacer recess of the multiple spacer recesses. In some such further exemplary embodiments, the multiple spacer recesses and movable non-rigid spacers are disposed at regular intervals around the first conical body. In still some further exemplary embodiments, the multiple spacer recesses and movable non-rigid spacers comprise at least a first pair of a spacer recess and a movable non-rigid spacer disposed closer to a distal side of the first conical body than a second pair of a spacer recess and a movable non-rigid spacer.

In an exemplary orthopedic device connection mechanism assembly, the first conical body and the second conical body are selected from a group of nested conical shapes consisting essentially of: cones, frustums of cones, elliptic cones, frustums of elliptic cones, portions thereof, combinations thereof, and permutations thereof.

An exemplary orthopedic medical device connection mechanism assembly comprises: a first orthopedic medical device comprising: a first polyhedral body, the first polyhedral body having a nesting outer surface, and an area defining a spacer recess, the spacer recess extending into the first polyhedral body and communicating with the nesting outer surface, and a movable non-rigid spacer disposed within the spacer recess, the movable non-rigid spacer having an internal end internally disposed from an external end, wherein the external end is adjacently disposed to the nesting outer surface; and a second orthopedic medical device comprising: a second polyhedral body, a porous inner surface internally disposed from a second body outer surface, wherein the porous inner surface defines a concave area configured to closely receive the nesting outer surface of the first polyhedral body in an assembled configuration.

In an exemplary orthopedic device connection mechanism assembly, the first polyhedral body and the second polyhedral body are selected from a group of nested polyhedral shapes consisting essentially of: pyramids, frustums of pyramids, wedges, prisms, cupolae, frustums of cupolae, portions thereof, combinations thereof, and permutations thereof. In some such exemplary embodiments, the group of nested polyhedral shapes further comprises polyhedral shapes having one or more rounded edges.

In an exemplary orthopedic device connection mechanism assembly, the first polyhedral body further defines multiple spacer recesses extending into the first polyhedral body and communicating with the nesting outer surface. In some exemplary embodiments, the orthopedic device connection mechanism assembly further comprises multiple movable non-rigid spacers, wherein each movable non-rigid spacer of the multiple movable non-rigid spacers is disposed within a spacer recess of the multiple spacer recesses. In some further exemplary embodiments, the multiple spacer recesses and movable non-rigid spacers are disposed at regular intervals around the first conical body. In yet some further exemplary embodiments, the multiple spacer recesses and movable non-rigid spacers comprise at least a first pair of a spacer recess and a movable non-rigid spacer disposed closer to a distal side of the first conical body than a second pair of a spacer recess and a movable non-rigid spacer.

An exemplary orthopedic medical device connection mechanism assembly comprises: a first orthopedic medical device comprising: a first dome body, the first dome body having a nesting outer surface, and an area defining a spacer recess, the spacer recess extending into the first dome body and communicating with the nesting outer surface, and a movable non-rigid spacer disposed within the spacer recess, the movable non-rigid spacer having an internal end internally disposed from an external end, wherein the external end is adjacently disposed to the nesting outer surface; and a second orthopedic medical device comprising: a second dome body configured to closely receive the first dome body, a porous inner surface internally disposed from a second body outer surface, wherein the porous inner surface defines a concave area configured to closely receive the first dome body in an assembled configuration.

In an exemplary orthopedic device connection mechanism assembly, the first dome body and the second dome body are selected from a group of nested dome shapes consisting essentially of: hemispheres, frustums of hemispheres, domes, frustums of domes, domes of spheroids, frustums of domes of spheroids, domes of ellipsoids, frustums of domes of ellipsoids, portions thereof, combinations thereof, and permutations thereof.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

What is claimed is:

1. An orthopedic medical device connection mechanism comprising:
a first orthopedic medical device comprising:
a proximal end proximally disposed from a distal end;
a first body disposed between the proximal end and the distal end, the first body having:
a nesting portion disposed between the proximal end and the distal end of the first body, the nesting portion having a nesting portion proximal side proximally disposed from a nesting portion distal side along a length, and a nesting portion outer surface extending along the length, the nesting outer surface defining a first nesting geometric shape,
an area defining a spacer recess, the spacer recess extending into the first body and communicating with the nesting portion outer surface, and
a movable non-rigid spacer disposed within the spacer recess, the movable non-rigid spacer having an internal end internally disposed from an external end, wherein the external end is adjacently disposed to the nesting portion outer surface; and
a second orthopedic medical device comprising:
a second body configured to be nested with the first body,
a porous inner surface internally disposed from a second body outer surface, the porous inner surface defining a second nesting geometric shape, the second nesting geometric shape being complimentary to the first nesting geometric shape, wherein the porous inner surface defines an plurality of pores, wherein the nesting portion outer surface of the first orthopedic medical device abuts the porous inner surface of the second orthopedic medical device in an assembled configuration, and wherein the external end of the moveable non-rigid spacer is received by a pore of the plurality of pores in the assembled configuration.

2. An orthopedic medical device connection mechanism assembly comprising:
a first orthopedic medical device comprising:
a first conical body, the first conical body having a nesting outer surface, and an area defining a spacer recess, the spacer recess extending into the first conical body and communicating with the nesting outer surface, and
a movable non-rigid spacer disposed within the spacer recess, the movable non-rigid spacer having an internal end internally disposed from an external end, wherein the external end is adjacently disposed to the nesting outer surface; and
a second orthopedic medical device comprising:
a second conical body configured to closely receive the first conical body, a porous inner surface internally disposed from a second conical body outer surface, wherein the porous inner surface defines a concave area.

3. The orthopedic device connection mechanism assembly of claim 2 further comprising an assembled configuration, wherein the first conical body is disposed within the concave area, wherein the first conical body is closely received by the second conical body in the concave area, and wherein the external end of the movable non-rigid spacer engages a pore defined by the porous inner surface in the assembled configuration.

4. The orthopedic device connection mechanism assembly of claim 2, wherein the first medical device is an inserter selected from the group consisting essentially of: a femoral cone inserter, a provisional orthopedic screw holder, an acetabular cup inserter, a glenoid inserter, an augment inserter, and a humeral cone inserter assembly.

5. The orthopedic device connection mechanism assembly of claim 2, wherein the second medical device is an implant selected from the group consisting essentially of: a tibial cone, a femoral cone, an acetabular cup, a glenoid cup, a tibial base implant, a femoral condylar implant, a talar implant, a scaphoid implant, a lunate implant, a metatarsal implant, a metacarpal implant, a phalangeal implant, a pelvic implant, a spinal implant, a mandibular implant, a humeral implant, a radial implant, an ulnar implant, or a scapular implant, components of any of the foregoing, trial implants of any of the foregoing, and components of trial implants of any of the foregoing.

6. The orthopedic device connection mechanism assembly of claim 2, wherein the first conical body further defines multiple spacer recesses extending into the first conical body, wherein each of the multiple spacer recesses communicates with the nesting outer surface.

7. The orthopedic device connection mechanism assembly of claim 6 further comprising multiple movable non-rigid spacers, wherein each movable non-rigid spacer of the multiple movable non-rigid spacers is disposed within a spacer recess of the multiple spacer recesses.

8. The orthopedic device connection mechanism assembly of claim 7, wherein the multiple spacer recesses and movable non-rigid spacers are disposed at regular intervals around the first conical body.

9. The orthopedic device connection mechanism assembly of claim 8, wherein the multiple spacer recesses and movable non-rigid spacers comprise at least a first pair of a spacer recess and a movable non-rigid spacer disposed closer to a distal side of the first conical body than a second pair of a spacer recess and a movable non-rigid spacer.

10. The orthopedic device connection mechanism assembly of claim 2, wherein the first conical body and the second conical body are selected from a group of nested conical shapes consisting essentially of: cones, frustums of cones, elliptic cones, frustums of elliptic cones, portions thereof, combinations thereof, and permutations thereof.

11. An orthopedic medical device connection mechanism assembly comprising:
a first orthopedic medical device comprising:
a first polyhedral body, the first polyhedral body having a nesting outer surface, and an area defining a spacer recess, the spacer recess extending into the first polyhedral body and communicating with the nesting outer surface, and
a movable non-rigid spacer disposed within the spacer recess, the movable non-rigid spacer having an internal end internally disposed from an external end, wherein the external end is adjacently disposed to the nesting outer surface; and
a second orthopedic medical device comprising:
a second polyhedral body,
a porous inner surface internally disposed from a second body outer surface, wherein the porous inner surface defines a concave area configured to closely receive the nesting outer surface of the first polyhedral body in an assembled configuration.

12. The orthopedic device connection mechanism assembly of claim 11, wherein the first polyhedral body and the second polyhedral body are selected from a group of nested polyhedral shapes consisting essentially of: pyramids, frustums of pyramids, wedges, prisms, cupolae, frustums of cupolae, portions thereof, combinations thereof, and permutations thereof.

13. The orthopedic device connection mechanism assembly of claim 12, wherein the group of nested polyhedral shapes further comprises polyhedral shapes having one or more rounded edges.

14. The orthopedic device connection mechanism assembly of claim 11, wherein the first medical device is an inserter selected from the group consisting essentially of: a femoral cone inserter, a provisional orthopedic screw holder, an acetabular cup inserter, a glenoid inserter, an augment inserter, and a humeral cone inserter assembly.

15. The orthopedic device connection mechanism assembly of claim 11, wherein the second medical device is an implant selected from the group consisting essentially of: a tibial cone, a femoral cone, an acetabular cup, a glenoid cup, a tibial base implant, a femoral condylar implant, a talar implant, a scaphoid implant, a lunate implant, a metatarsal implant, a metacarpal implant, a phalangeal implant, a pelvic implant, a spinal implant, a mandibular implant, a humeral implant, a radial implant, an ulnar implant, or a scapular implant, components of any of the foregoing, trial implants of any of the foregoing, and components of trial implants of any of the foregoing.

16. The orthopedic device connection mechanism assembly of claim 11, wherein the first polyhedral body further defines multiple spacer recesses extending into the first polyhedral body and communicating with the nesting outer surface.

17. The orthopedic device connection mechanism assembly of claim 16 further comprising multiple movable non-rigid spacers, wherein each movable non-rigid spacer of the multiple movable non-rigid spacers is disposed within a spacer recess of the multiple spacer recesses.

18. The orthopedic device connection mechanism assembly of claim 17, wherein the multiple spacer recesses and movable non-rigid spacers are disposed at regular intervals around the first conical body.

19. The orthopedic device connection mechanism assembly of claim 18, wherein the multiple spacer recesses and movable non-rigid spacers comprise at least a first pair of a spacer recess and a movable non-rigid spacer disposed closer to a distal side of the first conical body than a second pair of a spacer recess and a movable non-rigid spacer.
